(19) United States
(12) Patent Application Publication
(10) Pub. No.: US 2003/0108490 A1
(43) Pub. Date: Jun. 12, 2003

(54) Method and test kit for avoiding long-term failures in root canal treatments

(76) Inventors: Leo Tjaderhane, Oulu (FI); Jaana Harkonen-Wahlgren, Helsinki (FI); Timo Sorsa, Helsinki (FI); Tuula Salo, Oulu (FI)

Correspondence Address: BIRCH STEWART KOLASCH & BIRCH, PO BOX 747, FALLS CHURCH, VA, 22040-0747, US

(21) Appl. No.: 10/204,766

(22) PCT Filed: Feb. 23, 2001
(86) PCT No.: PCT/FI01/00184

(30) Foreign Application Priority Data
Feb. 23, 2000 (FI) 20000422

(51) Int. Cl. C12Q 1/37; A61K 7/28

(52) U.S. Cl. 424/50; 435/23

(57) ABSTRACT

The present invention discloses methods and test kits which are useful tools for the dentist and provide means for evaluating the periapical disease activity or inflammation and monitoring the need of continued or alternative medical treatment before finalizing the root treatment in teeth by measuring the presence or absence of MMPs in root canal exudates. The test kit is preferably constructed for chair-side use.

### THE TECHNICAL FIELD OF THE INVENTION

The present invention relates to biochemical or immunochemical methods and test kits based on the detection of MMPs for avoiding long-term failures in root canal treatments including loss of teeth, restorative structures as well as for decreasing the risk of loss of expensive conservative and/or prosthetic constructions by detecting the presence or absence of periapical disease activity in root canals and by demonstrating the presence of roentgenographically undetectable fractures and other disease-inducing factors in teeth before filling the root canal. Periapical inflammatory disease activities in root canals is evaluated by recording the absence or presence of matrix metalloproteinases (MMPs) in the root canals.

### THE BACKGROUND OF THE INVENTION

A method and test kit for diagnosing periodontal diseases from gingival sulcular fluid by determining active matrix metalloproteinase 8 (MMP-8) is described in U.S. Pat. No. 5,736,341. A periapical infection and inflammation is, however, a condition differing essentially from periodontal diseases. The area of the inflammation is located inside the jaw bone, and normally the only access to the inflammation is the root canal of the tooth.

The first step in the development of the periapical inflammation is a bacterial invasion of the dental pulp. Even though the dental pulp is considered to be immunocompetent, pulpitis eventually leads into pulpal necrosis, as the pulpal defensive reactions cannot resist constant bacterial invasion. During the invasion of the root canal system the bacteria initiate the defensive response in the bone surrounding the root apex. This response results into resorption of the surrounding bone, and formation of a granulomatous periapical tissue to prevent the spreading of the bacteria from the root canal into the bone. In a slow-progressing or chronic state of the periapical disease, the infection is restricted into the root canal system by the host's defensive mechanisms.

Chronic apical disease activities and apical granuloma, are inflammatory reactions of the periapical bone characterized by the presence of a granulomatous tissue predominantly infiltrated with lymphocytes, plasma cells and macrophages, and a well-developed fibrous capsule. Chronic periapical granuloma may be asymptomatic for years, and usually it does not expand rapidly. However, for reasons unknown at present, it may became acute with clinical manifestations such as abscess, apical exacerbation and fistula formation. This exacerbation may result in a rapid bone resorption and enlargement of the lesion. Apical granuloma may also develop into a periapical (odontogenic) cyst. In a cyst, an epithelium-lined cavity is formed, with the extraepithelial tissue and the collagenous capsule. The cysts may enlarge slowly, finally occupying large areas of the alveolar bone.

The presence of bacteria in the periapical lesions is a matter of controversy. Some authorities argue that the body's own defensive mechanisms take care of the bacteria present extraradicularly, some others believe that the occurrence of the bacteria is rare and confined only to the acute phases of the infection whereas others have demonstrated cultivable bacteria in most of the periapical granulomas and cysts. The evidence of the viable bacteria outside of the root, and especially the wide variety of the bacteria observed indicates that the extraradicular defensive reactions cannot be relied on and a diagnosis of periapical disease based on a bacterial method or test kit would not be sufficiently reliable.

The ultimate goal of endodontic treatment is the prevention or healing of the periapical lesion. According to cohort studies, the mean number of periodontal lesions per person approximate one in a population level. The number increases with increasing age and the proportion of affected teeth increases even more. It has been estimated that at the age of 60, 25% of the remaining teeth have been root filled. However, failure to eliminate the periapical inflammation is fairly common. Said being a good indication of the frequency of the condition as well as the need of good diagnostic tools.

The possible reasons for the unsuccessful outcome of the treatment are numerous. One of them is the failure to eliminate the existing periapical infection and inflammation. A considerable percentages of periapical pathologies fail to show improvement in the radiographic periapical status after one year. One prominent reason for the failures is the lack of means for evaluating the inflammatory status of the periapex prior to the completion of the treatment. The inflammatory status could not be controlled because no reliable diagnostic means for that kind of evaluation are available, at present.

Other reasons for the periapical lesions to remain after root canal treatment exist as well. Vertical root fractures may cause a leakage of oral bacteria into root canal system, thus allowing continuous recontamination. Vertical fractures may be much more common than suspected due to the difficulties in diagnosing minute fractures. Bacteria remaining in the root canal system after chemomechanical cleaning of the canals have been recognized as the most obvious cause. The presence of bacteria is essential for the periapical pathogenesis, but the bacteria may be located in inaccessible locations such as lateral canals, apical delta, dentinal tubules or root cementum. Even extraradicular infection is possible, as cultivable bacteria have been demonstrated in most of the periapical granulomas and cysts. In addition, bacterial culturing tests are expensive, time-consuming and require specific laboratories. Therefore, they cannot be regarded as standard clinical tests. However, regardless of their location, bacteria always induce inflammation leading into or causing the persistence of the periapical periodontitis lesion.

Chronic periapical periodontitis may remain asymptomatic and therefore unnoticed by the patient and the dentist for years, but is nonetheless a serious infectious and inflammatory condition. Unnoticed and therefore untreated oral infections, including periapical infections have been demonstrated to be a significant risk factor for fatal systemic diseases such as cardiovascular disease, arteriosclerosis and myocardial and cerebrovascular infarctions. Moreover, chronic subacute infections possess a continuous risk for patients with compromised immunological defensive system, such as diabetics or patients with rheumatoid arthritis. In addition, elimination of the periapical infection and inflammation is a prerequisite for a successful comprehensive treatment of the masticatory system as a whole, as the root canal treatment is eventually followed by the restorative treatment of the tooth, including prosthetic crowns and bridges. Failure to eliminate the periapical infection may therefore lead into loss of teeth and restorative structures with potentially marked financial consequences to the patient.

The diagnosis of the presence of the periapical inflammation is traditionally based on the radiographic examination and the presence of the inflammatory exudate in the root canals. Currently, there are no practical methods to estimate the activity of the inflammation in the periapex, or whether the root canal treatment procedures have resulted in a suppression of the inflammation. Therefore, the decision of completing the root canal treatment including filling the root canal and restorating the tooth, has been based on subjective estimation of the dentist. Because many causative factors of the presence or continuation of the periapical inflammation including incomplete root canal instrumentation, extraradicular infection or vertical root fracture are extremely difficult or impossible to be identified by the dentist, the failure to recognize the ongoing inflammation frequently results in filling the root canals in cases where appropriate healing has not or will not occur. Therefore, a considerable percentage of long-term failures occur after treatments. This, in turn, has caused a marked need for retreatments and surgical treatments, with additional costs and further decline in the percentage of successful treatment.

As a summary it is concluded that the periapical diseases consist of a group of inflammatory conditions, which, regardless of the presence or absence of the symptoms, require treatment of the root canal system. The aim of the treatment is the elimination of the infection of the root canal and the subsequent healing of the inflammation in the periapical bone. The importance of the initial endodontic treatment has been widely accepted. The success rate of the endodontic retreatments caused by the existing periapical lesions is far lower than that of the initial treatment. Moreover, existing restorations or prosthetic constructions are lost during the re-entry into the canal.

The methods of diagnosis currently available are useful in the diagnosis of the presence and location of the infection and periapical inflammation, but there are no diagnostic means available to evaluate the inflammatory status of the periapical lesion during the treatment, whether the medication seems to be effective and complete healing can be expected if the root canal treatment is completed.

The lack of diagnostic means to evaluate the elimination of infection and inflammation has lead into treatment protocols in which the root canals are obturated and the treatment completed without any informed knowledge of the potential to achieve healing of the periapical lesion. The protocols are based on the clinically observed finding that most of the periapical lesions heal with time. However, up to 36% of the radiographically observed lesions fail to show any improvement after one year follow-up, even when treated by a specialist. The impaired healing leads in many occasions into surgical intervention and/or loss of conservative or prosthodontic rehabilitation of the tooth crown. These treatments cause discomfort and additional costs to the patients.

As said above bacterial sampling from the root canal is an impractical and unreliable method to evaluate the success of endodontic therapy during the treatment. As a conclusion with the conventional methods currently in use the response of treatment cannot be monitored and in connection with an intervention the root canal, it cannot be determined if for example the medication in use should be continued and eventually changed. Therefore, there is a great need for a diagnostic system that would allow to estimate the disease activity prior to the final filling of the root canal system.

The objective of the present invention is to provide a biochemical, preferably an immunochemical chair-side method and test kit to evaluate the presence of the inflammatory status in the apical area of the tooth, which would allow the treating dentist to make an informed decision whether periapical healing may be expected and the tooth is ready for the completion of the treatment or if further medication is needed in order to obtain complete healing of the inflammation in the periapical area.

### A SHORT DESCRIPTION OF THE DRAWINGS

FIG. 1 depicts MMP-levels found in root canals.

FIG. 2 depicts patient data of MMP in root canals.

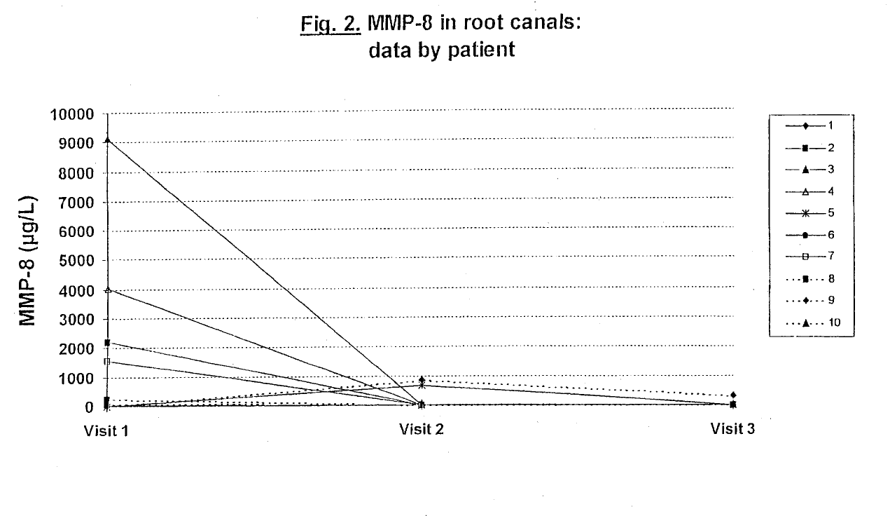

### THE SUMMARY OF THE INVENTION

The characteristic features of the present invention are defined in the claims.

### THE DETAILED DESCRIPTION OF THE INVENTION

In the following description, reference will be made to various methodologies known to those skilled in the art of immunochemistry and immunopathology, clinical chemistry, pharmaceutical sciences, dentistry and dental pathology. Publications and other material setting forth such known methodologies to which reference is made are incorporated herein by reference in their entireties as though set forth in full. General principles of immunoassays and the generation and use of antibodies as laboratory and clinical tools are set forth, for example, in Antibodies, A Laboratory Manual (Harlow, E. and Lane, D., eds., Cold Spring Harbor Laboratory, Cold Spring Harbor, N.Y., 1988) and in Principles and Practice of Immunoassay (C. P. Price and D. J. Newman (Eds.), Stockton Press, New York 1997).

Unless defined otherwise, all technical and scientific terms used herein have the same meaning as they commonly have in the art to which this invention belongs. In the description as follows, a number of terms used in immunology and dentistry are extensively used. In order to provide a clear and consistent understanding of the specification and claims, including the scope to be given such terms, the following definitions are provided.

Conventional Patient Examination

The diagnosis of the existing infection and inflammation in the periapical bone is based on the patient examination. Spontaneous pain and/or pain during mastication, sensitivity to percussion, tenderness in palpation of the periapical gingival tissues indicate an acute phase of the inflammation. The diagnosis is confirmed with periapical radiograph, which may also lead into diagnosis of a chronic periapical periodontitis even in the absence of symptoms. However, during early phases of the infection, the diagnosis is difficult because of the minute changes in the periapical bone and the inherent weaknesses of the radiological diagnosis, and the early inflammatory lesions may easily be overlooked. The differential diagnosis between active, progressive lesions and latent, non-progressive lesions are not reliable

The definite diagnosis can be made only after the access cavity has been prepared into the pulp chamber. Sensitivity during drilling, the presence of vital pulp, blood from the canal system at the time of the access preparation and during initial instrumentation confirm the diagnosis of pulpitis, the inflammation of the pulp tissue. When no vital pulp is present, and the root canal system contains necrotic tissue and/or pus, a diagnosis of gangrena or necrosis can be made. However, both vital tissue and necrotic canals can be present in the same tooth, especially in the multi-rooted teeth. As the periapical inflammatory reaction may be initiated with the vital pulp still present in the root canal system, the diagnosis of pulpitis does not exclude the possibility of periapical pathosis.

Conventional Bacterial Diagnosis

Pulpal infection is prerequisite for the periapical periodontitis to occur. Practically any oral bacteria can be pathogenic. In the root canals of the teeth with periapical lesions there usually is a mixed infection with at least 2-7 species, but also monoinfections can occur (especially Enterococcus faecalis).

During the invasion of the root canal system the bacteria initiate an inflammatory defensive response in the bone surrounding the root apex. This response results into resorption of the surrounding bone, and formation of the granulomatous periapical tissue to prevent the spreading of the bacteria from the root canal into the bone.

Bacterial sampling for the identification of the pathogens in a particular root canals may be performed in treatment-resistant pathological cases and to determine the antibiotic resistance of the bacteria causing the disease. While some species may be more pathogenic and cause more periapical tissue destruction than others, a variety of combinations of species have been indicated to cause severe periapical tissue destruction (Stashenko et al., Crit Rev Oral Biol Med 1998;9:498-521). Also, in untreated infected cases root canals may be filled with bacteria, while the periapical lesion may remain unchanged for several years. Therefore, bacterial sampling cannot be used to evaluate the disease activity and progression in the periapical area, nor the prognosis of the treatment. Moreover, bacterial analysis require sophisticated microbiological laboratory facilities, are expensive and above all time consuming. Thus, they cannot be regarded as a standard diagnostic procedure during the root canal treatment.

As a conclusion, the presently available methods aim at the making the diagnosis of the infectious and inflammatory process leading into the periapical pathosis. There are no reliable methods to evaluate the activity of the inflammatory process to help in the selection of treatment strategies or in the defining the prognosis of the treatment.

Treatment Strategies

Single-visit Technique

Single-visit endodontic treatment refers to the treatment, in which the root canals are cleaned, shaped and filled during only one visit of the patient. The single-visit technique has been adopted in order to reduce the possibility of inter-appointment contamination of the root canals, the suspected lower risk for the post-treatment pain, and the reduced chair-side time thus resulting into lower costs of the treatment. Therefore this technique has important advantages compared to the multiple-visit treatment. The case selection for the single-visit endodontic treatment possesses, however, a difficult task. It has been shown that after single-visit treatment the healing of the periapical lesion is markedly compromised when compared to the multiple-visit treatment (Trope et al., J Endod 1999;25:345-350). Therefore, a diagnostic tools for a more reliable patient selection are needed for the single-visit treatment protocol.

Multiple-visit Technique

The so-called multiple-visit technique in endodontic treatment aims at better disinfection of the root canal system by applying intracanal medication after the cleaning and shaping the root canals during the initial appointment. After filling the canals with medicament, the bacteria-tight seal is applied and after variable time—depending on the medication used—the root canals are reopened and either obturated, or the medication is renewed. It has been stated that in most cases there are no cultivable bacteria in the canals after 1-2 week medication with calcium hydroxide (Ca(OH)2).

The multiple-visit technique has several disadvantages. Even though the intracanal medication greatly enhances the elimination of bacteria from the root canal system, there are currently no medicament available that are effective against all possible pathogens. Therefore, the total elimination of bacteria cannot be ensured without bacteria sampling and culturing. As there are no practical chair-side methods to screen for the presence of bacteria in the canals, expensive and laborious microbiological culture methods are needed. Even if the bacteria sampling is performed, the presence of extraradicular infection cannot be ensured. In addition, temporary fillings may be lost between the appointments, resulting into contamination of the canals. The increased number of visits increase the costs of the treatments.

In some cases, the intracanal medication is continued for several months in order to observe the radiographic healing to occur before the completion of the treatment. While the prolonged radiographic observation with the intracanal medication may be the most effective method to ensure healing of the periapical lesion, it also causes a considerable delay in the completion of the treatment, and has not been widely accepted as a practical method for everyday practice. However, it has been shown that 26% of the teeth obturated after one week of disinfection with Ca(OH)2 failed to show any healing in the periapical lesion area after one year (Trope et al., J Endod 1999;25:345-350). The diagnostic tools to evaluate the ongoing inflammation in the periapical region would thus offer a possibility for an informed decision to be made about the effectiveness and duration of the intracanal medication needed for the onset of the healing.

The present invention is based on investigations which have shown that inflammation-related matrix metalloproteinases (MMPs) are present in the inflammatory infiltrate or exudate in the root canals and is related to methods and test kits for evaluating the activity of periapical inflammation of teeth. The evaluation is based on recording the presence or absence of matrix metalloproteinases.

Matrix Metalloproteinases in Periapical Lesions

During the periapical pathogenesis, the destruction of extracellular matrix of bone must occur in order to the lesion to form and enlarge. In inflammatory diseases, such as periodontitis, host matrix metalloproteinases (MMPs) are responsible for this degradation (Birkedal-Hansen, J. Periodontol 1993;64:474-484). MMPs include collagenolytic (collagenases) and gelatinolytic (gelatinases) enzymes, which together with other members of MMP family are capable of degrading practically all extracellular matrix components.

Because the presence and role of MMPs in the pathogenesis of periapical granulomas was not previously known, the present inventors started a research projects in order to study the presence, molecular species and quantity of MMP-8 in cases of pulpal inflammation and necrosis, using MMP-8 immunofluorometric assay (IFMA). They also analyzed the effect of root canal treatment (RCT) on the MMP-8 levels observed in root canals. Furthermore, the cells responsible for the liberation of MMP-8 in pulpal and periapical granulation tissue were examined by immunohistochemistry.

The principally known methods and test kits, which can be developed based on the present invention provide tools for a rapid chair-side test for avoiding long-term failures after root canal treatment by evaluation of the inflammatory status in teeth before completing the treatment and are useful for checking if the medical treatment has been effective or if there is a need for a prolonged treatment or changing treatment modalities before filling the root canals in order to eliminate the need of reopening the root canal due to undetected or not fully cured periapical inflammation. The method and test kit for excluding periapical disease is based on a per se known biochemical and/or immunochemical measurement of the presence of matrix metalloproteinases, particularly matrix metalloproteinase-8.

A chair-side method and test kits for diagnosing periodontitis by measuring active matrix metalloproteinase-8 from the gingival sulcular fluids is already available (U.S. Pat. No. 5,736,341). Basically the same method would be applicable to the diagnosis of the periapical inflammation, which is caused by the exposure of the tooth pulp to the bacteria by dental caries, crown or root fractures, or leaking restorations. The procedure of measuring the presence of matrix metalloproteinases (MMPs) in the root canal exudate is rapid, non-invasive, and easily performed during the routine root canal treatment from exudates discarded during the treatment. It does not require specific skills from the dentist, and except for the test kit, no additional instruments or chemicals are needed.

The best way to estimate the presence of the inflammatory reaction in the periapical region in the jawbone is to measure the presence and amount of MMPs in the root canal. In cases of persistent inflammation, the root canal exudate contains MMPs secreted by the inflammatory cells in the periapical region. If the root canal treatment procedures have been successful, the amount of MMPs in the exudate decrease rapidly. If the root canal treatment has not been successful for one reason or another, a continuous recordation of the presence of MMPs in the root canals would help the dentist to recognize the potential failure prior to finishing the treatment. It would also allow the dentist to make the decision of the further treatment needs for the tooth in question (for example, changing the root canal medication, extended root canal instrumentation, surgical treatment, or ultimately extraction of the tooth). The availability of an informed decision would therefore prevent the long-term failures of the root canal treatment, and thus prevent the loss of already fabricated expensive conservative or prosthetic structures in the clinical crown of the tooth. A further advantage of the present invention is the fact that the determination of MMPs in the root canal exudate can be carried out as a chair-side test which may be performed while the patient is in the dental office, e.g. in the dental operatory, even by unqualified dental office personnel.

Immunoassay in the present invention refers to a method or procedure capable of detecting and/or measuring a substance wherein the active and specific reagents include at least one binding substance or antibody capable of specifically binding MMPs or fragments thereof. Especially preferable, but not necessary are binding peptides which specifically recognize the active sites or epitopes of MMPs. Basic types of immunoassays include antigen capture assay, antibody capture assay and antibody sandwich assays.

The invention relates to both biochemical and/or immunochemical means and methods, but especially to immunological means and methods for evaluating the presence or absence of periapical disease and especially the inflammatory status in root canals. The means and methods based on the evaluation of presence or absence of MMPs was shown to provide a reliable chair-side test for assessing the inflammatory status in root canals. The evaluation of presence or absence of MMPs was shown to provide a reliable chair-side test for assessing the inflammatory status in root canals, being therefore a diagnostic aid in detecting conditions compromising the success of the root canal treatment such as roentgenographically undetectable minimal fractures or bacteria.

Biochemical MMP-tests can be developed based upon determination of enzymatic reactions. For example, collagenase activity can be measured as collagen degradation spectrophotometrically (227 nm) Lindy, S., et al., Eur. J. Biochem. 158, 1-4, 1986. The degradation of the synthetic peptide can also be monitored spectrophotometrically or fluorometrically (Tschesche, H., et al., In Methods in Enzymatic Analysis, Bergmeyer, U. H., ed., Verlag Chemie, Weinheim, Germany, pp 239-248, 1985). A multitude of biochemical methods for measuring MMPs (Suomalainen, Kimmo, Interstitial Collagenases in Gingival Crevicular Fluid and Saliva in Periodontal Diseases, Academic dissertation, Departement of Medical Chemistry and Department of Periodontology, University of Helsinki, Finland, Helsinki 1993) are in principle available but they are not as specific as immunochemical assays, e.g. due to false positives caused by enzymes released by bacteria. Thus, a biochemical marker test that detects periapical disease activity in a simple, practical and reliable manner requires sensitivity and specificity. Several methods havefor example been described to measure collagenolytic enzyme activity in saliva or gingival crevicular fluid. Generally, the activity is measured spectrophotometrically by observing the increase in absorbance caused by collagen degradation. Also, the degradation of a synthetic peptide as substrate connected to a color or fluorescence forming system can be followed spectrophotometrically or correspondingly, fluorometrically.

These methods, however, do not differentiate between MMPs of different origin. Because various enzyme activities of mammalian as well as bacterial origin are present can be present in an inflammed rrot canal, these biochemical methods are not sufficiently specific and often give inflated values. In spite of said disadvantage the present invention relates to test kits providing means to practice the methods of the invention using enzymatic reactions and/or binding substances, specifically binding or recognizing matrix metalloproteinases, such as antibodies including polyclonal and/or monoclonal antibodies or fragments thereof as well as binding peptides.

The immunochemical test kit according to the present invention may contain one or more antibodies, which recognize human MMPs, particularly human MMP-8 and by binding to said MMP forms a detectable agglutination. Alternatively the binding substances should be provided with at least one detectable labelled marker in a solid or liquid carrier.

The antibody of the invention is preferably monoclonal, especially the antibody that recognizes MMP-8. However, any antibody having the prerequisite characteristic, as described herein, is included. Methods for producing monoclonal antibodies, especially to active MMPs are described e.g. in the U.S. Pat. No. 5,736,341.

The test kit of the present invention can, in addition to the monoclonal antibody recognizing the mammalian MMPs contain at least one second binding substance which can be another monoclonal antibody, a polyclonal antibody or a binding peptide. When monoclonal, they are obtainable by using conventional hybridoma techniques, phage display techniques or recombinant DNA techniques.

Different types of test kits can be constructed to suit the immunological method which has been selected. Carrier materials and accessories are included in the test kit depending upon the method desired. The method is preferably chosen among immunochromatographic methods, immunometric methods, radioimmunoassays, radioimmunometric assays, enzyme immunoassays, fluoroimmunoassays, luminescence immunoassays, immmunoagglutination methods, hemagglutination methods, inhibition of agglutination methods and turbidimetric immunoassays. The detectable labels and optional carriers are selected according to the appropriate method. The most preferred test kits of the present invention for chair-side use are constructed according to immunochromatographic methods based on the lateral flow principle or an immunometric method based on the flow-through principle. A multitude of different immunotests performed on convenient test strips are well known in the art.

The method for evaluating the inflammatory status in the root canal, is essentially performed as an immunological assay including the following steps. A substantially non-invasive sample is collected from the opened root canal with a sampling device. Thin solid devices, such as paper-strips can be used for collecting site-specific samples from the opened root canal. In an optional embodiment of the invention the sampling device is also used as the test device. The sample is then placed in contact with at least one monoclonal antibody, which is already attached to the sampling or test device or the test device is alternatively dipped into a buffer solution containing the sample device from which the MMPs are extracted. Alternatively, the sampling device can be placed in contact with the test strip and a driving buffer added.

The construction of a test kit is described in more detail below, but it is not limit to the described test kit. Various representatives of poly- and monoclonal antibodies recognizing MMPs are listed below and can be produced by per se known methods. They are also commercially available for example from the following sources.

Monoclonal Antibodies

MMP-3: (A. F. Schuetzdeller Biochemicals; Triple Point Biologics; Research & Diagnostic Systems Inc.; Fuji Chemicals Limited; Oncogene Research Products; Calbiochem-Novabiochem Corp.; ICN Biomedicals); MMP-7: (A. F. Schuetzdeller Biochemicals; Research & Diagnostic Systems Inc.; MMP-9: Research & Diagnostic Systems Inc.; Fuji Chemicals Limited; Calbiochem-Novabiochem Corp.

Polyclonal Antibodies

MMP-1: Triple Point Biologics; MMP-8: (Triple Point Biologics); MMP-9: Biogenesis Ltd.; Chemicon International Inc.

Monoclonal antibodies of the present invention have been developed according to the original technique of Kohler and Milstein (Nature 256, 495, 1975). Methods for producing said antibodies recognizing MMP-8, especially in their active form are described in the U.S. Pat. No. 5,736,341, which is herewith incorporated by reference. Similarly, monoclonal antibodies recognizing other matrix metalloproteinase related molecules can be produced by those skilled in the art. As representatives of binding substances recognizing matrix metalloproteinase related molecules (MMP-RMs), those binding substances recognizing NGAL can be mentioned. Methods for their detection and production are described in the U.S. Pat. No. 5,866,432, which is also hereby incorporated by reference.

The antibodies can optionally be tagged with a label or marker molecule capable of making the presence or absence of the MMPs recordable. Various labels, markers or tags, also called tracer when combined with the antibodies or respective antigens, are known and described in literature, laboratory handbooks and patent publications. Such labels or markers are, for example, coloured latex particles, fluorochromes, liposomes, metal colloids, etc. However, it is to be noted that it is not necessary to use such labels and markers. In turbidimetric assays for example, only one polyclonal antibody is used. When binding to the antigen the sample solution gets turbidic. The turbidity is caused by antibody-antigen-aggregates forming during the reaction. Said aggregates can be detected visually.

Based on the results obtained and the antibodies available the present inventors used the test kit and methods described below for checking the inflammatory status before filling the root canal. The method and test kit was shown to be rapid, efficient and reliable.

Different numeric results which can be obtained in the experiments reflect the use of different sets of binding substances or antibodies as well as other variation in the test conditions, e.g. the size and dilution of the sample. However, it is possible to standardize the test kit and methods so that a +/− result indicates that the medication must be continued or the root canal can be filled for example using the single visit technique, frequently used in USA.

The results are even more reliable if applied to the two-visit or multiple visit technique frequently used in Europe.

The monoclonal antibodies specific to active human and/or mammalian MMP-8 developed according to the above procedure are used for designing a variety of test methods useful in the assessment of periapical disease activity. Both quantitative and qualitative methods can be developed.

A standard method for immunologically detecting the presence of MMPs is visually observing its agglutination when placed in contact with the antibody as such or coated on solid particles. The particles include latex particles. These latex particles can be coloured and used as tracers in immunochromatographic methods, wherein the coloured particles are allowed to move on a test strip placed on or in a solid support.

The periapical disease activity test as described herein can be based on the immunochromatographic principle. This technique, often referred to as the lateral flow technique, has been described in detail in EP 291 194 which is incorporated herein by reference. WO 94/15215 includes a test device that essentially consists of a membrane and an absorbing pad in a dipstick constructed with a chamber-like gap. In the immunometric version that employes two different antibodies, the first antibody is coated on particles that act as a visually detectable label or marker or by suitable instruments (fluorescent or chemiluminescent signal producing). The particles can be made, for example, of latex, colloidal metal (gold, selene) or a dispersing dye. These label particles are attached in a test device so that when the absorbing part of the device is brought into contact with the liquid sample and the sample is absorbed, the particles will migrate with the liquid flow and simultaneously, label antibody will bind the antigen (active MMP-8) if present in the sample. The liquid will be further absorbed into the membrane in the device. On the membrane, a second antibody (monoclonal or polyclonal antiMMP recognizing an epitope other than the first antibody) has been attached in a zone-like area. When the liquid flow carrying the label migrates through this zone, those label particles that have bound antigen will be bound to the zone. Thus, the zone will be detectable if there was antigen present in the sample.

This immunoassay technique can also be based on the use of one antibody only. This can be done by using antigen coated label particles in competition with antigen possibly present in the sample. The monoclonal antibody specific to the MMP is attached in a zone on the membrane. Sample antigen will occupy the antibody binding sites in the zone and thus, no detectable zone will appear.

Immunochromatography can also be made quantitative by measuring the signal produced by a label that is bound to the membrane when known standards or unknown samples are run. Visual semiquantitation is possible if several antibody zones with increasing antibody amount in the zone are used in the test device.

The above mentioned immunoassay techniques are useful for the development of a rapid chair-side test with a short performance time (often only a few minutes). The more recent techniques (lateral flow and flow-through) will provide tests that can be performed and interpreted very reliably by personnel untrained to laboratory work. They also lack some major disadvantages connected with agglutination methods, such as, for instance, false positives with samples containing rheumatoid factor and difficult interpretation of, especially, turbid samples.

For a site-specific dipstick test, the dentist can collect a specimen of root canal exudate fluid by placing a thin nitrocellulose sheet or a filter paper strip in contact with the root canal exudate. The strip is allowed to absorb the exudate, preferably for a standardized time. Then, the strip is transferred to a test tube with an adequate buffer solution where sample proteins are extracted. In case an immunochromatographic dipstick format is used, the dipstick is directly dipped into the tube for the test. Besides the filter strips other absorbing materials like porous plastics or ceramics as well as organic or inorganic silica compounds are also applicable, probably attached to a holder for convenient transfer. Liquid can also be collected in a capillary tube of glass or plastic. Finally, a dipstick-type test device can be so designed that it includes an absorbing end that is placed in the periapical pocket and the sample is absorbed directly into test device.

A site-specific dipstick test for evaluating the inflammatory status and ruling out the possibility of periapical disease in the individual site or directing the dentist to further studies need not be a quantitative test and is preferably a qualitative test. The threshold values or cut-off concentration for each individual method or test is calibrated and chosen by as such known methods by those skilled in the art so as to give optimal sensitivity and specificity. In a periapical disease activity test MMP concentrations, which cannot be recorded, should indicate that no periapical disease activity is present, which is an indication that healing is going on and that the root canal treatment can be completed without the risk of long-term failure. Especially, if two negative results, i.e. no MMP present, are obtained when applying the two visit technique, the result is very reliable. If the concentration is recordable or high it can be interpreted as an indication that the medication should be continued and the test repeated before filling the root canal, whereas a repeated high presence of MMPs is an indication that medication is probably not applicable and extraction or surgical intervention is the only remedy.

The feasibility and reliability of the method and test kits according to the present invention as compared to conventional methods is described in the following experiments.

Material and Methods

10 patients (11 teeth), with noncontributory medical histories, suffering from apical periodontitis, were selected for the collection of the root canal exudate samples. The diagnosis of the periapical periodontitis based on clinical symptoms and the x-ray examination, demonstrating the bone loss and disappearance of the periodontal ligament space in the periapical region. The sample collection was done with the approval of Ethical Committee of Helsinki University Faculty of Medicine, and with the patient's informed consent. The samples were collected during three consecutive appointments with two-week intervals, during which the root canal treatment was completed.

Primary access to the pulp and root canals was made according to regular treatment protocol. The instrumentation of the root canal was started with size 20 or 25 ISO file. X-ray was taken to determine the preparation length. At that time point, a sterile paper point was inserted into the root canal up to the preparation length for two minutes to absorb exudate from the root canal and periapical lesion. The root canals were then instrumented according to standard root canal treatment protocol. Finally, CaOH2 paste was inserted into the canal, and the access cavity was sealed.

After two weeks the root canals were reopened. After quick drying of the canals, a sterile paper point was inserted into the canal for two min for the sample collection. If the canal appeared dry, a drop of sterile saline was inserted to allow the absorption of the sample into the paper point. The root canal was then rinsed, dried and CaOH2 paste was inserted into the canal. After two weeks the procedure for the sample collection was repeated, and the root canals were filled with gutta percha and sealer in normal fashion.

Concentration of MMP-8 in the samples was determined from the elution buffer by a time-resolved immunofluorescence assay as described previously (Hanemaaijer et al., 1997). The monoclonal MMP-8 specific 8708 and 8706 antibodies were used as a catching antibody and tracer antibody, respectively. The tracer antibody was labeled using europium chelate. The assay buffer contained 20 mM Tris-HCl, pH 7.5, 0.5 M NaCl, 5 mM CaCl2, 50 μM ZnCl2, 0.5% bovine serum albumin, 0.5% sodium azide, and 20 mg/l DTPA. Samples were diluted in assay buffer and incubated for one h, followed by incubation for one h with tracer antibody. Enhancement solution was added, and after 5 min fluorescence was measured using a 1234 Delfia Research Fluorometer (Wallac, Turku, Finland).

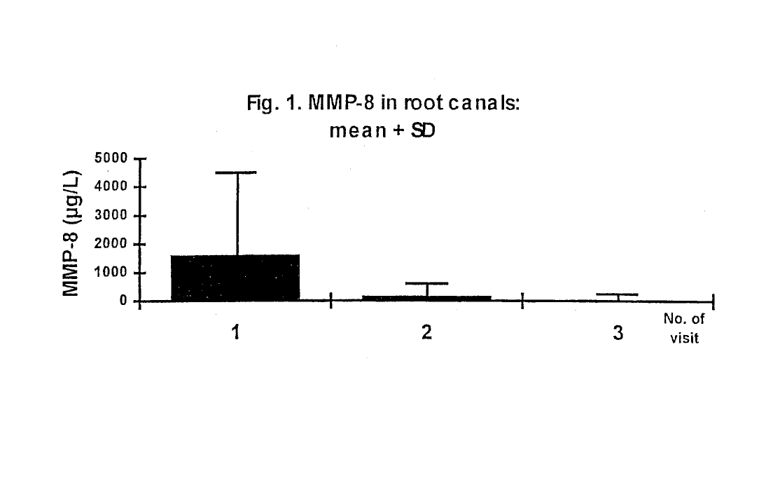

Kruskall-Wallis 1-way ANOVA was used to analyze the degree of difference in the MMP-8 levels between the different appointments within each sample site. Whenever a statistically significant difference was observed with Kruskall-Wallis test, Mann-Whitney U-test was used to determine which appointments displayed statistically significant difference. The MMP-8 levels markedly decreased during the root canal treatment of the necrotic teeth (FIG. 1). Analyzing the pooled data revealed a statistically significant decrease in the levels of MMP-8 between the 1st and the 3rd visits (Kruskall-Wallis p=0.02; Mann-Whitney U-test p=0.0107). However, even after removal of the pulpal tissue remnants and debris and a careful mechanical preparation of the root canals accompanied with the local medication with calcium hydroxide paste, a relatively high amount of MMP-8 was detected in the canals (FIG. 1). In the absence of the pulpal tissue the only possible source for MMP-8 in the canal is the periapical tissue. Only after the renewal of the local medication for fortnight was MMP-8 no longer detected in the root canals (FIG. 1).

When using the laboratory method described above tor measuring MMP-8, the data of each patient was observed separately, five teeth were found to express values over 1000 ng/ml during the first appointment when the treatment began. Interestingly, said 5/11 patients (or 36%) exactly corresponded to the failure rate of 36% in single-endodontic treatment cases (Trope et al., J Endod 1999;25:345-350). This indicates that by testing the presence of MMP-8 in the intraradicular exudate might serve as a tool to select the cases in which the single-visit technique would not result into healing of the periapical lesion.

In the second appointment, all the patients with initially high levels of MMP-8 in the exudate demonstrated undetectable levels of MMP-8 after two weeks of Ca(OH)2 treatment. In these cases, the testing indicates that the inflammation in the periapical lesion has subsided and the healing of the bone lesion is underway. Instead, two other patients with initial value of 0 demonstrated moderately increased levels (100-1000 ng/ml) of MMP-8 in the root canal samples. The levels above 100 ng/ml have been shown to indicate site-specific destructive inflammatory state in the periodontal gingival pockets (U.S. Pat. No. 5,736,341). Filling the root canals during that appointment might have caused a persisting infection-inflammation in the periapical area, with no radiographically observable healing during the regular follow-ups. The percentage (18%) is consistent with the one observed after one year follow-up in the patients with one-week intracanal Ca(OH)2 medication (24%) (Trope et al., J Endod 1999;25:345-350). The increase of this inflammatory enzyme may be due to the extrusion of the canal content into the periapical area during the instrumentation. This extrusion of infected root canal debris may then have caused an inflammatory response. With these patients, another two weeks with Ca(OH)2 was enough to subside the infection-inflammation in the periapical area for another patient, while the with the last one the level of MMP-8 did not reach zero, and alternative treatment methods may be needed to overcome the infection and inflammation in the periapical bone.

With one tooth the level of MMP-8 was extraordinarily high during the second visit (13800 ng/ml), and even in the third visit the MMP-8 level exceeded 1000 ng/ml. Later, after completing the treatment, the teeth had to be extracted and the root was found to be vertically fractured. This fracture could not be seen in X-ray photographs. Thus, a continued exceptionally high MMP-8 level seems to be a clear indication that treatment will not be efficient.

Even if the preliminary results obtained above are quantitative, the figures are non-binding as it is obvious to anyone skilled in the art, that the actual figures vary depending upon the size of sample, dilution, the type of binding substance, etc.

We claim:

1. Use of biochemical or immunochemical reagents capable of specifically recognizing matrix metalloproteinases (MMPs) for manufacturing a biochemical or immunochemical test kit for avoiding long term failure in root canal treatments including the loss of teeth, restorative structures, conservative and/or prosthetic constructions due to undetected periapical disease activity by determining the presence or absence of matrix metalloproteinase from a root canal exudate sample.

2. A method for avoiding long term failure in root canal treatments including decreasing the risk of loss of teeth, restorative structures, conservative and/or prosthetic constructions due to undetected periapical disease activity in root canals comprising the steps of:
(a) placing an exudate sample obtainable from an opened root canal in contact with a biochemical or immunochemical reagent capable of specifically recognizing at least one matrix metalloproteinase;
(b) determining the presence of mammalian matrix metalloproteinase with a biochemical or immunochemical assay capable of indicating a recordable reaction between said reagents and the matrix metalloproteinase present in the root canal exudate; and
(c) evaluating based on the recorded result the need of continued or alternative medical treatment before completing the root canal treatment.

3. The method according to claim 2, wherein the absence of matrix metalloproteinase indication of periapical disease healing or that progressing healing can be expected and the root canal treatment can be completed without continued medical treatment.

4. The method according to claim 2, wherein the presence of matrix metalloproteinase indicates that the periapical disease that the medical treatment should be continued before completing the root canal treatment.

5. The method according to claim 2, wherein a repeatedly occuring presence of matrix metalloproteinase is an indication of roentgenographically undetectable minimal fracture or other disease-inducing factors which cannot be healed by medication.

6. The method according to claim 5, wherein a continued presence of matrix metalloproteinase is an indication that extraction or surgical intervention of the tooth is the treatment of choice.

7. The method according to claim 2, wherein the matrix metalloproteinase is MMP-8.

8. The method according to claim 2, wherein the immunochemical reagent is a binding substance.

9. The method according to claim 2, wherein the biochemical reagent is a substrate for a enzymatic reaction.
